(19) United States
(12) Patent Application Publication
(10) Pub. No.: US 2025/0205087 A1
(43) Pub. Date: Jun. 26, 2025

(54) WELDING HEADGEAR WITH HEAD STRAP TIGHTNESS FEEDBACK AND LIMITING

(71) Applicant: Illinois Tool Works Inc., Glenview, IL (US)

(72) Inventor: Praveen Dandu, Green Bay, WI (US)

(21) Appl. No.: 18/969,978

(22) Filed: Dec. 5, 2024

Related U.S. Application Data
(60) Provisional application No. 63/612,865, filed on Dec. 20, 2023.

(51) Int. Cl. A61F 9/06; A42B 3/08

(52) U.S. Cl. CPC A61F 9/06; A42B 3/085

(57) ABSTRACT

Systems and methods are provided for welding headgear with head strap tightness feedback and limiting. An example welding-type headgear includes a head strap component configured for engaging user's head, and a tightening mechanism configured for setting or adjusting tightness of the head strap component on the user's head, with the tightening mechanism including at least one tightening control component for enabling the setting or adjusting of the tightness of the head strap component. The tightening mechanism is configured to perform one or both of, providing feedback to indicate, when the tightness of the head strap component reaches a pre-set value, that the pre-set value is reached, and limiting, after the tightness of the head strap component reaches the pre-set value, the setting or adjusting the tightness of the head strap component via the at least one tightening control component to the pre-set value.

### CLAIM OF PRIORITY

This patent application claims priority to and claims benefit from U.S. Provisional Patent Application Ser. No. 63/612,865, filed on Dec. 20, 2023. The above identified application is hereby incorporated herein by reference in its entirety.

### BACKGROUND

Welding has become increasingly ubiquitous. Welding can be performed in an automated manner or in a manual manner (e.g., being performed by a human). Various equipment or components may be used during welding operations. For example, welding helmets (or other similar welding headgear) are sometimes worn by welders (operators or users) when performing welding operations. The welding helmets may have a hard shell configured to shield the head of an operator from welding spatter, and a viewing window so that the operator can see the surrounding environment while wearing the helmet. The viewing window may be incorporated into a moveable part. In some instances, conventional welding solutions may have some limitations and/or disadvantages. For example, conventional welding helmets may have limitations and/or disadvantages with respect to ease of use, comfort, and the like.

Further limitations and disadvantages of conventional approaches will become apparent to one skilled in the art, through comparison of such approaches with some aspects of the present systems and methods set forth in the remainder of this disclosure with reference to the drawings.

### BRIEF SUMMARY

Aspects of the present disclosure relate to welding solutions. More specifically, various implementations in accordance with the present disclosure are directed to systems and methods for welding headgear with head strap tightness feedback and limiting, substantially as illustrated by or described in connection with at least one of the figures, and as set forth more completely in the claims.

These and other advantages, aspects and novel features of the present disclosure, as well as details of an illustrated implementation thereof, will be more fully understood from the following description and drawings.

### BRIEF DESCRIPTION OF THE DRAWINGS

FIG. 1 shows an example welding-type setup that may be used for welding-type operations.

FIG. 2 shows an example welding-type headgear incorporating head strap tightening mechanism with feedback, in accordance with the present disclosure.

FIG. 3 shows an example welding-type headgear incorporating head strap tightening mechanism with feedback, limiting, and release, in accordance with the present disclosure.

FIG. 4 shows an example head strap component of a welding-type headgear, in accordance with the present disclosure.

### DETAILED DESCRIPTION

As utilized herein, the terms “circuits” and “circuitry” refer to physical electronic components (e.g., hardware), and any software and/or firmware (“code”) that may configure the hardware, be executed by the hardware, and/or otherwise be associated with the hardware. As used herein, for example, a particular processor and memory (e.g., a volatile or non-volatile memory device, a general computer-readable medium, etc.) may comprise a first “circuit” when executing a first one or more lines of code and may comprise a second “circuit” when executing a second one or more lines of code. Additionally, a circuit may comprise analog and/or digital circuitry. Such circuitry may operate, for example, on analog and/or digital signals. It should be understood that a circuit may be in a single device or chip, on a single motherboard, in a single chassis, in a plurality of enclosures at a single geographical location, in a plurality of enclosures distributed over a plurality of geographical locations, etc. Similarly, the term “module” may, for example, refer to a physical electronic components (e.g., hardware) and any software and/or firmware (“code”) that may configure the hardware, be executed by the hardware, and/or otherwise be associated with the hardware.

As utilized herein, circuitry or module is “operable” to perform a function whenever the circuitry or module comprises the necessary hardware and code (if any is necessary) to perform the function, regardless of whether performance of the function is disabled or not (e.g., by a user-configurable setting, factory trim, etc.).

As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the term “exemplary” means serving as a non-limiting example, instance, or illustration. As utilized herein, the terms “for example” and “e.g.” set off lists of one or more non-limiting examples, instances, or illustrations.

Welding-type power, as used herein, refers to power suitable for welding, plasma cutting, induction heating, CAC-A (carbon arc cutting/air) and/or hot wire welding/preheating (including laser welding and laser cladding). Welding-type power supply, as used herein, refers to a power supply that can provide welding-type power. A welding-type power supply may include power generation components (e.g., engines, generators, etc.) and/or power conversion circuitry to convert primary power (e.g., engine-driven power generation, mains power, etc.) to welding-type power.

Welding-type operations, as used herein, comprise operations in accordance with any known welding technique, including flame welding techniques such as oxy-fuel welding, electric welding techniques such as shielded metal arc welding (e.g., stick welding), metal inert gas welding (MIG), tungsten inert gas welding (TIG), resistance welding, as well as gouging (e.g., carbon arc gouging), cutting (e.g., plasma cutting), brazing, induction heating, soldering, and/or the like.

Welding-type setup, as used herein, refers to any setup comprising welding related devices or equipment (e.g., welding power sources, welding torch, welding gear such as headwear and the like, auxiliary devices or systems, etc.) which are used in facilitating and/or in conjunction with welding-type operations.

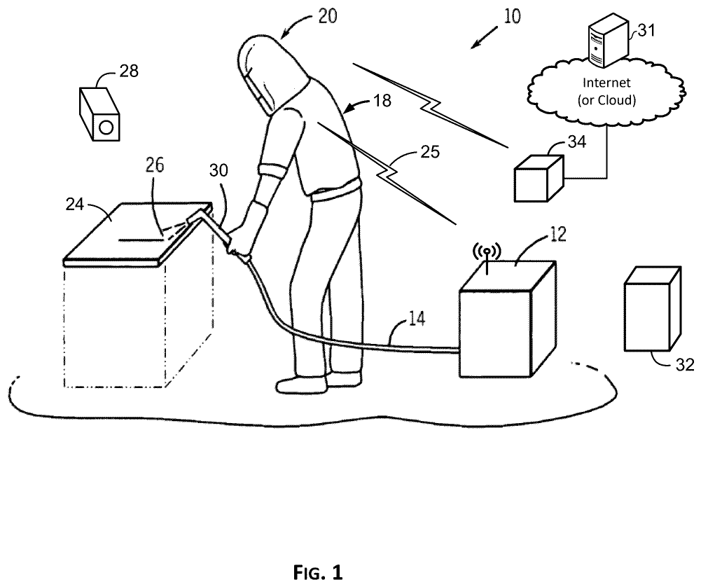

FIG. 1 shows an example welding-type setup that may be used for welding-type operations. Referring to FIG. 1, there is shown an example welding-type setup 10 in which an operator (user) 18 is wearing welding headwear 20 and welding a workpiece 24 using a torch 30 to which power is delivered by equipment 12 via a conduit 14, with weld monitoring equipment 28, which may be available for use in monitoring welding operations.

The equipment 12 may comprise a power source, optionally a source of a shielding gas and, where wire/filler material is to be provided automatically, a wire feeder. Further, in some instances an engine 32 may be used to drive equipment or components used during welding operations. The engine 32 may comprise a gas engine or a liquefied petroleum (LP) engine. The engine 32 may drive generators, power sources, etc. used during welding operations.

The welding-type setup 10 of FIG. 1 may be configured to form a weld joint by any known welding-type technique. For example, optionally in any implementation, the welding equipment 12 may be arc welding equipment that provides a direct current (DC) or alternating current (AC) to a consumable or non-consumable electrode of a torch 30. The electrode delivers the current to the point of welding on the workpiece 24. In the welding-type setup 10, the operator 18 controls the location and operation of the electrode by manipulating the torch 30 and triggering the starting and stopping of the current flow. In other implementations, a robot or automated fixture may control the position of the electrode and/or may send operating parameters or trigger commands to the welding system. When current is flowing, an arc 26 is developed between the electrode and the workpiece 24. The conduit 14 and the electrode thus deliver current and voltage sufficient to create the electric arc 26 between the electrode and the workpiece. The arc 26 locally melts the workpiece 24 and welding wire or rod supplied to the weld joint (the electrode in the case of a consumable electrode or a separate wire or rod in the case of a non-consumable electrode) at the point of welding between electrode and the workpiece 24, thereby forming a weld joint when the metal cools.

Optionally in any implementation, the weld monitoring equipment 28 may be used to monitor welding operations. The weld monitoring equipment 28 may be used to monitor various aspects of welding operations, particularly in real-time (that is as welding is taking place). For example, the weld monitoring equipment 28 may be operable to monitor arc characteristics such as length, current, voltage, frequency, variation, and instability. Data obtained from the weld monitoring may be used (e.g., by the operator 18 and/or by an automated quality control system) to ensure proper welding.

As shown, the equipment 12 and headwear 20 may communicate via a link 25 via which the headwear 20 may control settings of the equipment 12 and/or the equipment 12 may provide information about its settings to the headwear 20. Although a wireless link is shown, the link may be wireless, wired, or optical.

Optionally in any implementation, equipment or components used during welding operations may be driven using engines. For example, the engine 32 may drive generators, power sources, etc. used during welding operations. In some instances, it may be desired to obtain information relating to used engines. For example, data relating to engines (and operations thereof) used during welding operations may be collected and used (e.g., based on analysis thereof) in monitoring and optimizing operations of these engines. The collection and use of such data may be performed telematically—that is, the data may be collected locally, subjected to at least some processing locally (e.g., formatting, etc.), and then may be communicated to remote management entities (e.g., centralized management locations, engine providers, etc.), using wireless technologies (e.g., cellular, satellite, etc.).

Optionally in any implementation, a dedicated controller (e.g., shown as element 34 in FIG. 1) may be used to control, centralize, and/or optimize data handling operations. The controller 34 may comprise suitable circuitry, hardware, software, or any combination thereof for use in performing various aspects of the engine related data handling operations. For example, the controller 34 may be operable to interface with the engine 32 to obtain data related thereto. The controller 34 may track or obtain welding related data (e.g., from weld monitoring equipment 28, from equipment 12, etc.). The controller 34 may then transmit the data (e.g., both engine related and weld related data), such as to facilitate remote monitoring and/or management, by way of wireless communications. This may be done using cellular and or satellite telematics hardware, for example.

In some example implementations, welding-type systems or setups, such as the welding-type setup 10, may be configured for collecting and reporting data relating to welding-type operations and/or to functions or components utilized during welding-type operations. For example, data from welding processes, power sources, welding-related accessories etc. in a weld setup may be collected. In this regard, the collected data may comprise, for example, current, voltage, wire feed speed, weld states, and numerous other power source parameters and settings.

The collected data may then be sent to remote entities (e.g., a remote server 31, which may be a manufacturer-controlled, Internet-based cloud server) and/or to local systems or devices (e.g., local PC, a tablet, a smartphone, etc.). The collected data may be utilized in enhancing welding-related systems and/or operations. For example, manufacturers may utilize the collected data to identify issues (and correct them) and/or devise modifications or improvements in the various components. Further, users may be able to generate reports on collected data to measure, document, and improve their processes.

Improving or enhancing operation of the various components of welding-type setups, such as the welding-type setup 10 of FIG. 1, is desirable. For example, users may wear or use welding-type headgear (e.g., the welding headwear 20 in FIG. 1) when performing welding-type operations in welding-type setups. Enhancing ease of use and reliability of welding-type headgear may improve performance of welding-type setups as a whole, and of welding-type operations performed therein. Such enhancement may be achieved by addressing shortcomings in existing welding-type headgear and/or by adding features not currently available, especially where such features are added in cost effective manner. Conventional solutions may be improved upon by, e.g., enhancing features and/or functions relating to the securing of the welding-type headgear (e.g., onto the user's head).

For example, welding-type headgear (e.g., welding helmets) may incorporate components (e.g., head strap) for engaging user's head, to secure the welding-type headgear thereto. When using welding-type headgear, users may tighten the head strap, such as by turning a dial or a knob, to secure the welding-type headgear to their heads. This tightness keeps the welding-type headgear secured onto the user's head when pulling up/down the welding mask (or shield) to cover their face while welding. The dial allows adjusting tightness of head strap based on preference. Use of such head straps may have some limitations and challenges, however. For example, a user may not know how tight is “tight enough” when trying to tighten the head strap, and as such may not tighten the head strap enough or may tighten it too much. When the head strap is over-tightened it may cause discomfort while using or damage their forehead. If head strap is not tightened enough, the welding-type headgear may slide or fall off completely, which may cause injury or bad welds if that happens while welding.

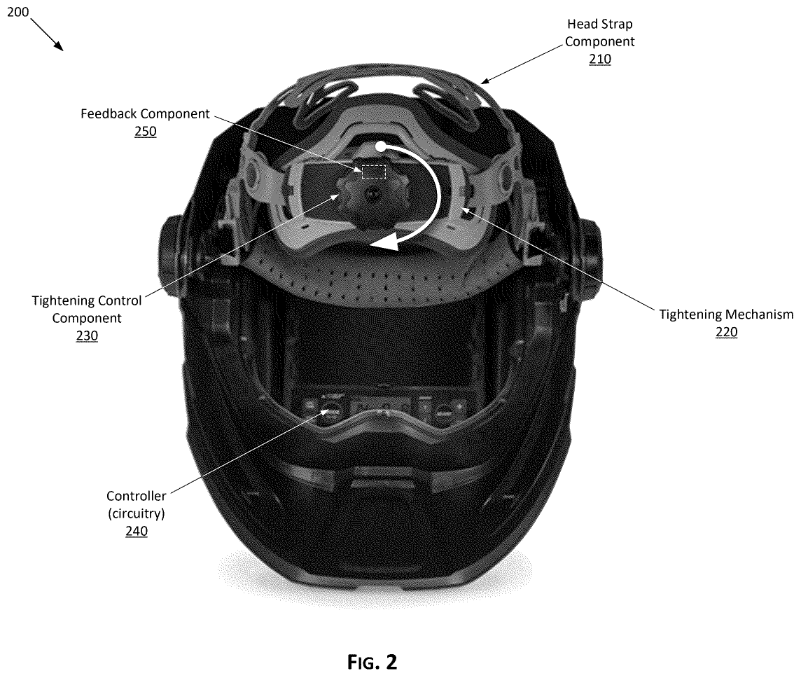

Solutions based on the present disclosure address some of the limitations and/or challenges that may arise with convention solutions, especially with respect to tightening of head straps (or similar components) of welding-type headgear. In this regard, in various implementations, welding-type headgear may be configured such that when head straps (or similar components) thereof are tightened enough, feedback (e.g., audible, haptic, or the like) is provided to indicate to the user that the head strap had been tightened enough. This may be done to prevent over-tightening. As such, a pre-set value(s) may be set or selected, for use as threshold in determining when the tightening is enough. Additionally, or alternatively, further setting or adjusting of tightness of the head strap may be limited once the pre-set value(s) is reached. The same approach or design may be used for under-tightening—e.g., where feedback may be provided if the tightening is insufficient. In some implementations additional features may be used, such as auto-tightening, tightness release (or reduction), and the like, and the welding-type headgear may be configured to support these features. Example embodiments incorporating solutions and features based on the present disclosure are illustrated and described in more detail below, particularly with respect to FIGS. 2-4.

FIG. 2 shows an example welding-type headgear incorporating head strap tightening mechanism with feedback, in accordance with the present disclosure. Shown in FIG. 2 is welding-type headgear (e.g., welding helmet) 200.

The welding-type headgear 200 may be any suitable welding-type headgear configured for use in welding-type operations, particularly being worn by the welder (user) to provide protection thereof. The welding-type headgear 200 may be welding helmet as known in the art. In this regard, in various implementations, the welding-type headgear 200 may comprise a hard (blower) shell configured to shield the head of an operator during welding operations (e.g., from welding spatter, and the like), and a viewing window so that the operator may be able see the surrounding environment while wearing the helmet. In some instances, the viewing window may be incorporated into a moveable part, such as a welding mask (or shield). As such, users may be able to expose their faces without having to remove the entire helmet. In this regard, the welding mask may be rotatable so that the user may raise the welding mask away without removing the entire welding-type headgear 200. Thus, by rotating the welding mask, users may have allowed access to the face while keeping the shell of the welding-type headgear stationery. The welding mask may comprise a vision protection part (or viewing window), through which the use may look during the welding-type operation. The vision protection part may comprise a darkening lens or auto-darkening filter (ADF) to protect the user's vision from the brightness of the welding arc. In some instances, welding-type headgear 200 may include an interior welding mask or the like, which may have little or no darkening capability, and which the user may use in lower-light environments to provide protection against other conditions associated with welding-type operations—e.g., sparks, flying debris, etc. Nonetheless, it should be understood that the disclosure is not limited to any particular type or design of welding-type headgear.

In some instances, welding-type headgears (such as the welding-type headgear 200) may incorporate suitable circuitry configured for use in supporting use of welding-type headgears, and/or various features or functions associated therewith. This may include suitable circuitry for controlling operation of certain components (e.g., controlling amount of darkening via the vision protection part), for supporting communication functions (e.g., communication with other devices or systems in the welding-type setup), to provide or support input/output (I/O) functions, etc. For example, as illustrated in FIG. 2, the welding-type headgear 200 comprises a controller circuitry 240.

In some instances, welding-type headgears (such as the welding-type headgear 200) may incorporate a component configured for securing the welding-type headgear to the user's head. For example, as illustrated in FIG. 2, the welding-type headgear 200 comprises a head strap component 210. The head strap component 210 may be configured for engaging the user's head, and securing the welding-type headgear 200 thereto. The head strap component 210 may be configured such that it may be adjustable, where it may be loose in an initial state, and then once placed onto the user's head, the user may tighten the head strap component 210 to secure the welding-type headgear 200 onto the user's head. To that end, the head strap component 210 may incorporate or otherwise be coupled to tightening mechanisms (e.g., the tightening mechanism 220 in FIG. 2), which may be used to set or adjust the tightness of the head strap component 210 (relative to the user's head). In this regard, various designs or techniques may be used to effectuate the tightening of the head strap, and the disclosure is not limited to any particular approach, and as such any suitable design or technique may be used.

In some instances, tightening control components may be used to control or otherwise effectuate adjusting the tightness of the head strap, such as by modifying or otherwise controlling the tightening mechanism. For example, as shown in FIG. 2, the welding-type headgear 200 incorporates a tightening control component 230, which may be used in setting or adjusting the tightness of the head strap component 210. The tightening control component 230 may be incorporated into, or coupled to the tightening mechanism 220, but the disclosure is not so limited, and any suitable placement may be used. Further, various designs or techniques may be used in implementing the tightening control component(s), and the disclosure is not limited to any particular approach, and as such any suitable design or technique may be used. For example, as illustrated in FIG. 2, the tightening control component 230 uses a knob or dial based design, with the setting or adjusting of the tightness being done by rotating the knob or dial.

In accordance with the present disclosure, welding-type headgears (such as the welding-type headgear 200) may be configured to improve functions and/or operation thereof, particularly with respect to tightness of the head strap (or similar components) used therein. In this regard, as noted conventional welding-type headgear may have some limitations and shortcomings with respect to the tightness related features therein, particularly with respect to preventing over-tightening or under-tightening of the head straps (or similar components). Solutions based on the present disclosure may reduce the risk of over-tightening (which may cause discomfort or even injury, such as, e.g., headaches, forehead damage, etc.), and/or under-tightening, which results in the welding-type headgear being too loose, thus causing to slide and/or fall off (which may cause injuries or interruption during welding).

In particular, in various implementations based on the present disclosure, such issues may be addressed by, e.g., configuring the welding-type headgear to provide feedback or some indication to warn the user when the head strap (or similar component) is too tight or too loose, and/or to limit or otherwise override setting or adjusting the tightness of the head strap under certain conditions. In this regard, the disclosure is not limited to any approach, and any suitable type or form of feedback or indication may be used. For example, the feedback may comprise one or more of haptic feedback, audible feedback, or visual feedback. In knob or dial based implementations, such as the tightening control component 230 of FIG. 2, providing the feedback may comprise generating a clicking sound by the knob-based assembly in response to continued rotating of the knob when the tightening (or loosening) of the head strap reaches pre-set value(s). However, the disclosure is not limited to such approach, and as such in some instances, dedicated feedback component(s) may be used. This may include both existing feedback components (e.g., existing speaker, screen, etc.), and/or new feedback components integrated into the welding-type headgear for providing the required feedback.

In some instances, in addition to (or in lieu of) providing feedback, the welding-type headgear may be configured to limit the setting or adjusting of the tightness of the head strap component, after the tightness of the head strap component reaches the pre-set value(s). In this regard, as used herein “limiting” may not necessarily entail preventing the user from applying input for attempting to adjust the tightness of the head strap component; rather, it merely indicates that no further adjustment would occur—e.g., in knob or dial based implementations, after reaching the pre-set value(s), the user may still try to adjust the tightness (by rotating the knob or dial), but there would be no actual adjustment—e.g., due to slippage.

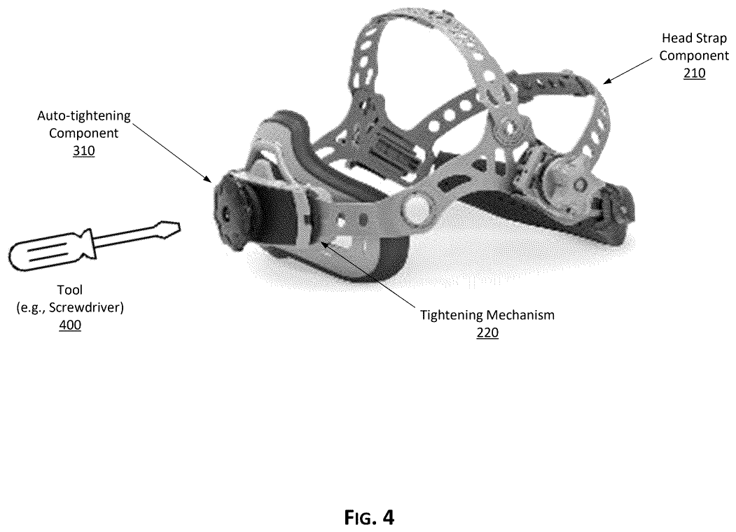

The pre-set value(s) may be determined in various ways, such as experimentally (e.g., during design and/or manufacturing of the welding-type headgear, or after manufacturing). As such, the pre-set value(s) may be a factory setting, or may be set after manufacturing (e.g., by the user or someone else). Further, in some instances, the pre-set value(s) may be adjusted by the user. Adjusting of the pre-set value(s) is described in more detail with respect to FIG. 4.

In some instances, the welding-type headgear may comprise sensors configured to generate sensory information relating to the tightness of the head strap component, which may be used in conjunction with the tightness related features as described herein. In this regard, the disclosure is not limited to particular type(s) and/or placement(s) of sensors, and any suitable type and/or placement of sensor may be used. Example sensors that may be used may comprise torque sensors, force sensors, pressure sensors, strain gauge sensors, etc., or any combination thereof. The sensory information may be fed into suitable circuitry which may be configured to utilize the sensory information in determining the tightness and/or in determining when pre-set value(s) may be reached. In this regard, existing circuitry (e.g., controller circuitry 240) may be used, or, alternatively, dedicated circuity may be added into the welding-type headgear for providing the required processing and use of the sensory information. The circuitry may use other data and/or other sources, in addition to or in lieu of sensory information, in determining (calculating) the tightness and/or determining when the pre-set value(s) are reached. For example, circuitry may be configured to make such determination(s) based on monitoring of the tightening control component 230.

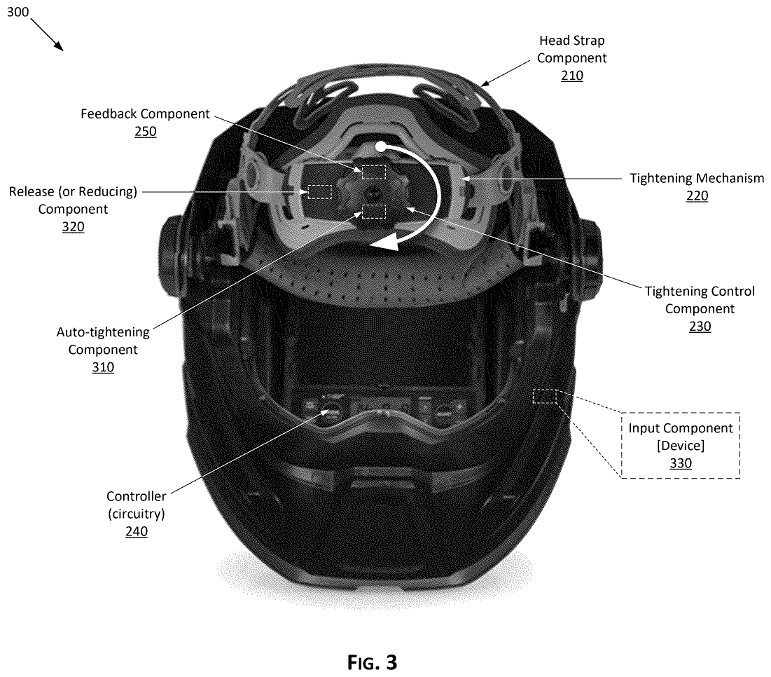

In some instances, additional features and/or functions may be used, such as auto-tightening, tightness release (or reduction), etc. Such features and functions are described in more detail with respect to FIG. 3.

FIG. 3 shows an example welding-type headgear incorporating head strap tightening mechanism with feedback, limiting, and release, in accordance with the present disclosure. Shown in FIG. 3 is welding-type headgear (e.g., welding helmet) 300.

The welding-type headgear 300 may be substantially similar to the welding-type headgear 200 of FIG. 2, and as such may comprises similar components and/or may be configured to operate in similar manner as welding-type headgear 200, and as such may be similarly configured to operate and/or provide tightening feedback and/or control related features based on the present disclosure, as described with respect to FIG. 2.

However, the welding-type headgear 300 may be further configured to provide or support additional tightening related features and/or functions. For example, the welding-type headgear 300 may be configured to support or provide auto-tightening functions. In this regard, the welding-type headgear 300 may be configured to enable automatically setting the tightness of the head strap component, such as to one of the pre-set value(s). This may be done, e.g., in response to user input. In this regard, the welding-type headgear 300 may comprise an auto-tightening component 310 configured to automatically set the tightness of the head strap component, such in response to the user input. For example, auto-tightening component 310 may automatically set the tightness of the head strap component to one of the pre-set value(s) in response to user input.

The welding-type headgear 300 may be also configured to support or enable releasing (or reducing) at least a portion of the tightness of the head strap. In this regard, as used herein “release” (or releasing) or “reduce” (or reducing) may comprise decreasing the tightness of the head strap (as currently set) by at least value or amount, which may include a complete release—that is, completely loosening the head strap. For example, welding-type headgear 300 may comprise an auto-release component 330 configured for enabling releasing or reducing the tightness of the head strap. The auto-release component 330 may be a dedicated (newly added) component, may be an existing component (e.g., a tightening control component, such as the tightening control component 230 in the headgear 300) configured to enable auto-release related functions. The value or amount by which the tightness may be reduced or released may be adaptively set or selected. The auto-release component 330 may also be used in setting or adjusting the release or reduction amount—e.g., based on interaction with the component, and/or characteristics or parameters associated therein (e.g., duration). The interaction may be done in any suitable manner, such as based on type of component used. For example, in knob or dial based implementations, the interaction may be done by pushing or pulling the knob or dial to reduce at least a portion of the tightness of the head strap component in response to user input, with the amount of release or reduction being governed by duration of pushing or pulling.

The welding-type headgear 300 may be also configured to support or enable receiving user input relating to the tightness related features or functions. For example, welding-type headgear 300 may comprise an input component (device) 330 configured for enabling applying user input relating to controlling the tightening mechanism and/or functions of the tightening mechanism as described herein.

FIG. 4 shows an example head strap component of a welding-type headgear, in accordance with the present disclosure. Shown in FIG. 4 is the head strap component 210 as described herein.

The head strap component 210 may be used in welding-type headgears, such as the welding-type headgear 200 or welding-type headgear 300), and may be particularly configured to incorporate and/or use features based on the present disclosure, such as providing feedback relating to the tightness of the head strap component 210, auto-tightening of the head strap component 210, and/or tightening release (or reduction), as described with respect to FIGS. 2-3. In this regard, as noted, a pre-set value(s) may be used in conjunction with such features, such as threshold for triggering providing the tightness-related feedback and/or the limiting of limiting of further setting or adjusting of the tightness.

In some instances, the pre-set value(s) is adjustable. In this regard, the adjusting of pre-set value(s) may be done in response to user input. As such, the welding-type headgear (and/or the head strap component 210 and the tightening mechanism 220 thereof in particular) may be configured to enable adjusting the pre-set value(s) in response to the user input. In this regard, the user input may be applied in different ways. For example, in some instances, the user input may be applied by use of a suitable tool 400 (e.g., screwdriver or the like, as shown in FIG. 4), and the head strap component 210 (and/or the tightening mechanism 220 thereof in particular) may be configured to enable applying the adjustment in that manner.

In the example use case scenario illustrated in FIG. 4, e.g., in which the tightening control component 230 may be a knob or dial based assembly, the pre-set value(s) may be adjusted (increased or decreased) may be adjusted by rotating a screw in the tightening control component 230 (e.g., clockwise or counterclockwise) using the screwdriver 400. Nonetheless, it should be understood that the disclosure is not limited to particular implementation or approach illustrated in FIG. 4, and that any suitable approach for adjusting the pre-set value(s) may be used.

Accordingly, solutions based on the present disclosure may yield various advantages over any existing conventional solutions. In this regard, with the features and functions incorporated into the welding-type headgear based on the present disclosure, the welding-type headgear may stay on the user's head on with sufficient tightness, and may provide new users and regular users a way to know exactly how much to tighten their welding-type headgear. This may be done while also accommodating more experienced users, by offering adjustability of the tightness pre-set values so that such experienced users may set customized tightness. Further, auto-release features may allow for quick-release, and thus removal of the welding-type headgear, if desired.

An example welding-type system, in accordance with present disclosure, comprises a welding-type headgear configured for use by a user during welding-type operation, the welding-type headgear comprising a head strap component configured for engaging user's head, and a tightening mechanism configured for setting or adjusting tightness of the head strap component on the user's head, with the tightening mechanism comprises at least one tightening control component for enabling the setting or adjusting of the tightness of the head strap component. The tightening mechanism is configured to perform one or both of: providing, when the tightness of the head strap component reaches a pre-set value, feedback to indicate that the pre-set value is reached; and limiting, after the tightness of the head strap component reaches the pre-set value, the setting or adjusting the tightness of the head strap component via the at least one tightening control component to the pre-set value.

In an example embodiment, the welding-type headgear further comprises a feedback component configured to generate and/or output the feedback.

In an example embodiment, the feedback comprises one or more of haptic feedback, audible feedback, or visual feedback.

In an example embodiment, the welding-type headgear further comprises one or more sensors configured to generate sensory information relating to the tightness of the head strap component. In this regard, the disclosure is not limited to particular type(s) and/or placement(s) of sensors, and any suitable type and/or placement of sensor may be used.

In an example embodiment, the one or more sensors comprise at least one of a torque sensor, a force sensor, a pressure sensor, and strain gauge sensor.

In an example embodiment, the pre-set value is pre-determined and pre-programmed into the tightening mechanism. In this regard, as used herein “pre-programmed” (or pre-programming) does not necessarily suggest or is limited to software programming, and may also include hardware programming—e.g., pre-configuring various hardware components based on selected or determined performance parameters or criteria.

In an example embodiment, the pre-set value is selected from a plurality of pre-set values.

In an example embodiment, the pre-set value is adjustable.

In an example embodiment, the tightening mechanism is configured to enable adjusting the pre-set value in response to user input.

In an example embodiment, the tightening mechanism is configured to enable applying the user input by use of a suitable tool.

In an example embodiment, welding-type headgear further comprises an input component configured for enabling applying user input relating to controlling the tightening mechanism and/or functions of the tightening mechanism.

In an example embodiment, the welding-type headgear further comprises a controller comprising one or more circuits configured to monitor the tightness of the head strap component and/or to determine when the tightness of the head strap component reaches the pre-set value.

In an example embodiment, the controller is configured to determine when the tightness of the head strap component reaches the pre-set value based on one or both of sensory information and monitoring of the at least one tightening control component.

In an example embodiment, the tightening mechanism is configured to enable overriding the limiting of the setting or adjusting of the tightness of the head strap component via the at least one tightening control component.

In an example embodiment, the at least one tightening control component comprises a dial based assembly.

In an example embodiment, the tightening mechanism is configured to limit the setting or adjusting the tightness of the head strap component by causing the dial to slip after reaching the pre-set value.

In an example embodiment, providing the feedback comprises generating a clicking sound by the dial based assembly after the tightness of the head strap component reaches the pre-set value.

In an example embodiment, the tightening mechanism comprises one or more reducing components configured to reduce at least a portion of the tightness of the head strap component in response to user input.

In an example embodiment, the tightening mechanism comprises one or more auto-tightening components configured to automatically set the tightness of the head strap component.

In an example embodiment, the one or more auto-tightening components are configured to automatically set the tightness of the head strap component to the pre-set value in response to user input.

In an example embodiment, the welding-type headgear comprises a welding helmet.

Other implementations in accordance with the present disclosure may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, various implementations in accordance with the present disclosure may be realized in hardware, software, or a combination of hardware and software. The present disclosure may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various implementations in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain implementations, it will be understood by those skilled in the art that various changes may be made and equivalents may be substituted without departing from the scope of the present disclosure. For example, block and/or components of disclosed examples may be combined, divided, re-arranged, and/or otherwise modified. In addition, many modifications may be made to adapt a particular situation or material to the teachings of the present disclosure without departing from its scope. Therefore, it is intended that the present disclosure not be limited to the particular implementation disclosed, but that the present disclosure will include all implementations falling within the scope of the appended claims.

What is claimed is:

1. A welding-type system, comprising:
a welding-type headgear configured for use by a user during welding-type operation, the welding-type headgear comprising:
a head strap component configured for engaging user's head; and
a tightening mechanism configured for setting or adjusting tightness of the head strap component on the user's head;
wherein the tightening mechanism comprises at least one tightening control component for enabling the setting or adjusting of the tightness of the head strap component; and
wherein the tightening mechanism is configured to perform one or both of:
providing, when the tightness of the head strap component reaches a pre-set value, feedback to indicate that the pre-set value is reached; and
limiting, after the tightness of the head strap component reaches the pre-set value, the setting or adjusting the tightness of the head strap component via the at least one tightening control component to the pre-set value.

2. The welding-type system according to claim 1, wherein the welding-type headgear comprises a feedback component configured to generate and/or output the feedback.

3. The welding-type system according to claim 1, wherein the feedback comprises one or more of haptic feedback, audible feedback, or visual feedback.

4. The welding-type system according to claim 1, wherein the welding-type headgear further comprises one or more sensors configured to generate sensory information relating to the tightness of the head strap component.

5. The welding-type system according to claim 4, wherein the one or more sensors comprise at least one of a torque sensor, a force sensor, a pressure sensor, and strain gauge sensor.

6. The welding-type system according to claim 1, wherein the pre-set value is pre-determined and pre-programmed into the tightening mechanism.

7. The welding-type system according to claim 6, wherein the pre-set value is selected from a plurality of pre-set values.

8. The welding-type system according to claim 1, wherein the pre-set value is adjustable.

9. The welding-type system according to claim 8, wherein the tightening mechanism is configured to enable adjusting the pre-set value in response to user input.

10. The welding-type system according to claim 9, wherein the tightening mechanism is configured to enable applying the user input by use of a suitable tool.

11. The welding-type system according to claim 1, wherein welding-type headgear further comprises an input component configured for enabling applying user input relating to controlling the tightening mechanism and/or functions of the tightening mechanism.

12. The welding-type system according to claim 1, wherein the welding-type headgear further comprises a controller comprising one or more circuits configured to monitor the tightness of the head strap component and/or to determine when the tightness of the head strap component reaches the pre-set value.

13. The welding-type system according to claim 12, wherein the controller is configured to determine when the tightness of the head strap component reaches the pre-set value based on one or both of sensory information and monitoring of the at least one tightening control component.

14. The welding-type system according to claim 1, wherein the tightening mechanism is configured to enable overriding the limiting of the setting or adjusting of the tightness of the head strap component via the at least one tightening control component.

15. The welding-type system according to claim 1, wherein the at least one tightening control component comprises a dial based assembly.

16. The welding-type system according to claim 15, wherein the tightening mechanism is configured to limit the setting or adjusting the tightness of the head strap component by causing the dial to slip after reaching the pre-set value.

17. The welding-type system according to claim 15, wherein providing the feedback comprises generating a clicking sound by the dial based assembly after the tightness of the head strap component reaches the pre-set value.

18. The welding-type system according to claim 1, wherein the tightening mechanism comprises one or more reducing components configured to reduce at least a portion of the tightness of the head strap component in response to user input.

19. The welding-type system according to claim 1, wherein the tightening mechanism comprises one or more auto-tightening components configured to automatically set the tightness of the head strap component.

20. The welding-type system according to claim 19, wherein the one or more auto-tightening components are configured to automatically set the tightness of the head strap component to the pre-set value in response to user input.

21. The welding-type system according to claim 1, wherein the welding-type headgear comprises a welding helmet.
